(19) United States
(12) Patent Application Publication
(10) Pub. No.: US 2022/0378625 A1
(43) Pub. Date: Dec. 1, 2022

(54) ABSORBENT ARTICLE COMPRISING AN INTERMEDIATE LAYER

(71) Applicant: The Procter & Gamble Company, Cincinnati, OH (US)

(72) Inventors: Gueltekin ERDEM, Beijing (CN); Yi Yuan, Beijing (CN); Jinyu Li, Beijing (CN); Hui Liu, Beijing (CN); Ernesto Gabriel Bianchi, Oberursel (DE); Abhishek Prakash Surushe, Kelkheim (DE); Sascha Kreisel, Frankfurt (DE)

(73) Assignee: The Procter & Gamble Company, Cincinnati, OH (US)

(21) Appl. No.: 17/826,201

(22) Filed: May 27, 2022

(30) Foreign Application Priority Data
Jun. 1, 2021 (CN) PCT/CN2021/097695

(51) Int. Cl. A61F 13/15; A61F 13/511; A61F 13/537

(52) U.S. Cl. CPC A61F 13/15203; A61F 13/5116; A61F 13/537; A61F 2013/530481

(57) ABSTRACT

The present disclosure relates to an absorbent article comprising a topsheet, a backsheet, and a layer of absorbent material disposed between the topsheet and the backsheet, wherein the layer of absorbent material comprises superabsorbent polymer, and an intermediate layer comprising a nonwoven web. The intermediate layer is disposed between the layer of absorbent material and the backsheet, wherein the intermediate layer has a MD tensile/basis weight no greater than about 0.75 N/5 cm/g/m² as measured according to Tensile Strength Test, and a thickness/basis weight no less than about 0.078 mm/g/m², as measured FTT Test.

### FIELD

The present disclosure relates to absorbent articles for personal hygiene that are worn in the crotch region of the wearer, for example baby diapers, training pants and adult incontinence products which are particularly thin, flexible, comfortable and having a soft feel.

### BACKGROUND

Disposable absorbent articles such as diapers and adult incontinence products are well known in the art. Such disposable articles are designed to absorb and contain body exudates, in particular large quantity of urine. These absorbent articles comprise several layers, for example a topsheet, a backsheet and in-between an absorbent core, among other layers.

One function of an absorbent core is to absorb and retain the bodily exudates for a prolonged amount of time, for example, overnight for a diaper, minimize re-wet to keep the wearer dry, and avoid soiling of clothes or bed sheets. Some currently marketed absorbent articles comprise absorbent cores comprising an absorbent material which is a blend of comminuted wood pulp (i.e., airfelt) with superabsorbent polymers (SAP) in particulate form, also called absorbent gelling materials. Absorbent articles having a core consisting essentially of SAP as the absorbent material (so called “airfelt-free” cores) have also been proposed. Majority of absorbent cores comprise an absorbent material at least partially wrapped with a core wrap.

Meanwhile, softness, flexibility and/or cushiony feel of the absorbent article are some of prioritized sensory requirements.

In some absorbent articles, especially in absorbent articles having a lower basis weight and/or a high percentage of SAP, in particular a particle form of SAP, a negative touch feeling known as “graininess” coming from the SAP particles needs to be improved. There have been several attempts to reduce a grainy feel including increase of a basis weight of a soft nonwoven layer at most outside of the article which the consumers can directly touch. However, increase of a basis weight of a soft nonwoven at most outside of an article brings increase in production cost as a soft nonwoven is relatively expensive. In addition, users' directly contact of the outermost soft nonwoven may bring the negative of “fuzz” at the outer nonwoven. Employment of an intermediate layer between the absorbent core and the backsheet of the absorbent article is another approach to mitigate the grainy feel. However, adding more materials to an absorbent article also makes the absorbent article stiffer which is not desirable by consumers.

Based on the foregoing, there is a need for an absorbent article with improvement in grainy feel without compromising flexibility of the absorbent article.

There is also a need for an absorbent article providing an improved softness while keeping the cost of manufacturing as low as possible.

### SUMMARY

The present disclosure relates to an absorbent article comprising a topsheet, a backsheet, and a layer of absorbent material disposed between the topsheet and the backsheet wherein the layer of absorbent material comprises superabsorbent polymer, and an intermediate layer comprising a nonwoven web, the intermediate layer being disposed between the layer of absorbent material and the backsheet, wherein the intermediate layer has a MD tensile/basis weight no greater than about 0.75 N/5 cm/g/m² as measured according to Tensile Strength Test, and a thickness/basis weight no less than about 0.078 mm/g/m², as measured FTT Test.

The present disclosure also relates to a nonwoven comprising a MD tensile/basis weight no greater than about 0.75 N/5 cm/g/m² as measured according to Tensile Strength Test, and a thickness/basis weight no less than about 0.078 mm/g/m², as measured FTT Test.

The present disclosure also relates to processes for producing nonwoven comprising supplying a nonwoven web comprising first thermoplastic fibers having a first melting temperature and second thermoplastic fibers having a second melting temperature, wherein the second melting temperature is at least about 40° C. higher than the first melting temperature and, applying heat to the nonwoven web so that at least part of the first thermoplastic fibers are heat-fused one another, wherein the heat has a temperature between the first melting temperature and lower than the second melting temperature.

### BRIEF DESCRIPTION OF THE DRAWINGS

In the drawings, like numerals or other designations designate like features throughout the views.

FIG. 1 is schematic plan view of an exemplary absorbent article according to the present disclosure.

FIG. 2A is a transversal cross-section of the diaper of FIG. 1 showing an intermediate layer between and in direct contact with a lower substrate layer and the backsheet.

FIG. 2B is a transversal cross-section of the diaper of FIG. 1, showing an intermediate layer between and in direct contact with the layer of absorbent material and a lower substrate layer.

FIG. 2C is a transversal cross-section of still another alternative diaper showing the intermediate layer between and in direct contact with the layer of absorbent material and the backsheet.

FIG. 3 is a schematic drawing of a through air bonder.

FIG. 4A is an SEM image of a cross-sectional view of Nonwoven 1.

FIG. 4B is another SEM image of a cross-sectional view of Nonwoven 1.

FIG. 5 illustrates an apparatus used in the Modified Fluid Acquisition Test.

FIG. 6A is a side view of the curved component used in the Modified Fluid Acquisition Test.

FIG. 6B is an end view of the curved component of FIG. 6A.

FIG. 6C is a bottom view of the curved component of FIG. 6A.

FIG. 6D is a bottom perspective view of the curved component of FIG. 6A.

FIG. 6E is a top perspective view of the curved component of FIG. 6A.

FIG. 7A illustrates a top plate assembly used in the Modified Fluid Acquisition Test.

FIG. 7B illustrates equipment used in the Modified Fluid Acquisition Test.

### DETAILED DESCRIPTION

Various non-limiting forms of the present disclosure will now be described to provide an overall understanding of the principles of the structure, function, manufacture, and use of an absorbent article comprising fastening members having unique shape. One or more examples of these non-limiting embodiments are illustrated in the accompanying drawings. Those ordinary skilled in the art will understand that the absorbent articles described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

As used herein, the term “absorbent article” refers to disposable devices such as infant, child, or adult diapers, adult incontinence products, training pants, sanitary napkins and the like which are placed against or in proximity to a body of a wearer to absorb and contain the various fluids (urine, menses, and/or runny BM) or bodily exudates (generally solid BM) discharged from the body. Typically, these absorbent articles comprise a topsheet, backsheet, an absorbent core, leg cuffs, optionally an acquisition system and/or a distribution system (which may be comprised of one or several layers), and typically other components, with the absorbent core normally placed at least partially between the backsheet and the acquisition and/or distribution system or between the topsheet and the backsheet. The absorbent articles comprising a fastening member of the present disclosure will be further illustrated in the below description and in the Figs in the form of one or more components of taped diaper. Nothing in this description should be, however, considered limiting the scope of the claims. As such the present disclosure applies to any suitable form of absorbent articles (e.g., diapers, training pants, adult incontinence products, sanitary napkins).

The term “air permeability” is defined by the Air Permeability Test set forth below. Air permeability is set forth in m³/m²/minute (m/min).

The term “joined”, “bonded”, or “attached” encompasses configurations whereby an element is directly secured to another element by affixing the element directly to the other element, and configurations whereby an element is indirectly secured to another element by affixing the element to intermediate member(s) which in turn are affixed to the other element.

The term “lateral” (and forms thereof), with respect to a line lying in a plane substantially occupied by an absorbent article fastening member laid flat and horizontally, viewed from above, relates to a direction substantially perpendicular to a longitudinal axis of the absorbent article.

The term “longitudinal” and “length” (and forms thereof), with respect to a line lying in a plane substantially occupied by an absorbent article fastening member laid flat and horizontally, viewed from above, relates to a direction approximately aligned with the wearer's spine when the article would be normally worn, with the wearer in a standing or extended reclining position.

The term “nonwoven” means a porous, fibrous material made from continuous (long) filaments (fibers) and/or discontinuous (short) filaments (fibers) by processes such as, for example, spunbonding, meltblowing, airlaying, carding, coforming, hydroentangling, and the like. Nonwovens do not have a woven or knitted filament pattern. Nonwovens may be liquid permeable or impermeable.

The term “staple fiber(s)” as used herein means an elongate particulate having a length less than 5.08 cm and/or less than 3.81 cm and/or less than 2.54 cm. The terms “web” or “web of material” refer to a sheet-like structure such as a nonwoven or a film.

Absorbent Article

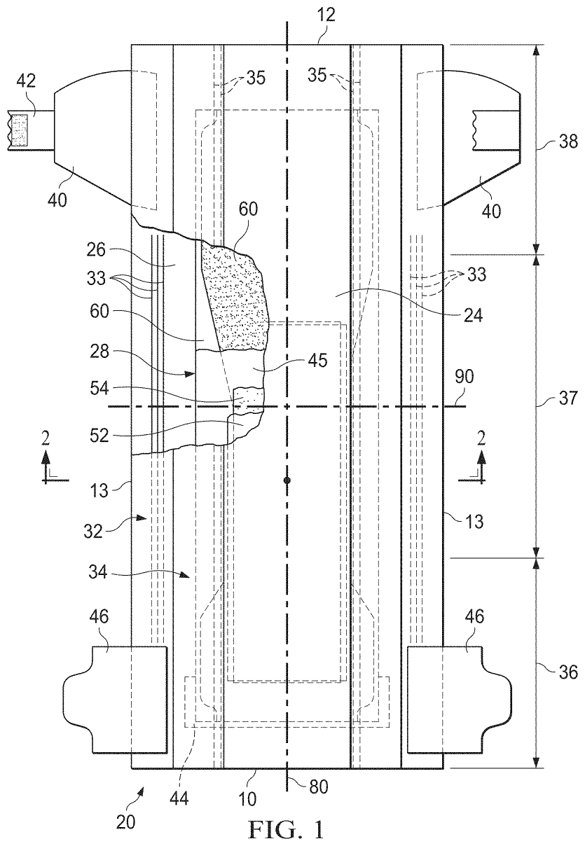

FIG. 1 is a plan view of an exemplary diaper 20, in a flat-out state, with portions of the diaper being cut-away to more clearly show the construction of the diaper 20. As said, this diaper 20 is shown for illustration purpose only as the structure of the present disclosure may be comprised in a wide variety of diapers or other absorbent articles, such as pants.

As shown in FIG. 1, the absorbent article, here a diaper, comprises a topsheet 24, backsheet 26, and a layer of absorbent material 28 which is positioned between the topsheet 24 and the backsheet 26. The layer of absorbent material 28 can absorb and contain liquid received by the absorbent article. The absorbent article of the present disclosure, such as the diaper 20 illustrated in FIG. 1, comprises an intermediate layer and may also comprise an upper acquisition and distribution system (ADS) 50. The upper ADS may comprise an upper 52 and lower 54 layer.

The absorbent article may also comprise barrier leg cuffs 34 and may further comprise elasticized leg cuffs 32. Moreover, the absorbent article may comprise a fastening system, such as an adhesive fastening system or a hook and loop fastening member, which can comprise tape tabs 42, such as adhesive tape tabs or tape tabs comprising hook elements, cooperating with a landing zone 44 (e.g. a nonwoven web providing loops in a hook and loop fastening system).

An absorbent article, such as the diaper 20 shown in FIG. 1 can be notionally divided in a first waist region 36 (which may be the front waist region), a second waist region 38 (which may be the back waist region) opposed to the first waist region 36 and a crotch region 37 located between the first waist region 36 and the second waist region 38. The longitudinal centerline 80 is the imaginary line separating the diaper along its length in two equal halves. The transversal centerline 90 is the imagery line perpendicular to the longitudinal line 80 in the plane of the flattened-out diaper and going through the middle of the length of the diaper (the same applies to for the transversal centerline and longitudinal line of other absorbent articles of the present disclosure). The periphery of the diaper 20 is defined by the outer edges of the diaper 20. The longitudinal edges 13 of the diaper may run generally parallel to the longitudinal centerline 80 of the diaper 20 and the end edges (the front waist edge 10 and the back waist edge 12) run between the longitudinal edges generally parallel to the transversal centerline 90 of the diaper 20. The crotch region, the first and the second waist region each constitute 1/3 of the absorbent article along the longitudinal centerline.

Further, the absorbent article may comprise other elements, such as a back waist feature, which may be non-elastic or elastic, and a front waist feature, which may be non-elastic or elastic, a lotion applied onto the body-facing surface of the topsheet, back ears 40, and/or front ears 46.

The front and/or back ears 40, 46 may be separate components attached to the absorbent article or may instead be continuous with portions of the topsheet and/or backsheet such that these portions form all or a part of the front and/or back ears 40, 46. Also combinations of the aforementioned are possible, such that the front and/or back ears 40, 46 are formed by portions of the topsheet and/or backsheet while additional materials are attached to form the overall front and/or back ears 40, 46. The front and/or back ears may be elastic or non-elastic. Also, the front ears 40 may be applied as separate components attached to the absorbent article while the back ears (or parts thereof) 46 may be continuous with portions of the backsheet and/or topsheet — or vice versa.

The topsheet 24 is the part of the absorbent article 10 that is in contact with the wearer's skin. The topsheet 24 may be joined to portions of the backsheet 26, the layer of absorbent material 28, to an upper nonwoven core web overlaying the layer of absorbent material towards the topsheet, to the barrier leg cuffs 32, and/or to any other layers as is known to those of ordinary skill in the art. At least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polypropylene or bicomponent PE/PP fibers or mixtures thereof), or a combination of natural and synthetic fibers. The topsheet may have one or more layers. The topsheet may be apertured or non-apertured, and may have any suitable three-dimensional features, and/or may have a plurality of embossments (e.g., a bond pattern). Any portion of the topsheet may be coated with a skin care composition, an antibacterial agent, a surfactant, and/or other beneficial agents.

The backsheet 26 is generally that portion of the absorbent article 20 that constitutes all or a part of the garment-facing surface of the absorbent article. The backsheet 26 may be joined to portions of the topsheet 24, the layer of absorbent material 28 and/or the intermediate layer (or to the layer of the intermediate layer that is in direct contact with the backsheet and/or any other layers of the absorbent article by any attachment methods known to those of skill in the art. The backsheet 26 prevents, or at least inhibits, the bodily exudates absorbed and contained in the layer of absorbent material 28 from soiling articles such as bedsheets, undergarments, and/or clothing. The backsheet is typically liquid impermeable, or at least substantially liquid impermeable.

The backsheet may, for example, be or comprise a thin plastic film 39, such as a thermoplastic film. Other suitable backsheet materials may include breathable materials which permit vapors to escape from the absorbent article, while still preventing, or at least inhibiting, bodily exudates from passing through the backsheet.

The backsheet may comprise a wetness indicator.

The backsheet 26 may comprise a backsheet outer cover nonwoven web 40. The backsheet outer cover nonwoven web may comprise one or more nonwoven materials joined to a backsheet film 39 and that covers the backsheet film 39. The outer cover nonwoven web 40 may form the garment-facing surface of the backsheet. Thereby, film may not be present on the garment-facing surface. The backsheet outer cover nonwoven web 40 may comprise a bond pattern, apertures, and/or three-dimensional features.

The layer of absorbent material 28 comprises superabsorbent polymer, such as superabsorbent polymer particles, and may optionally comprise cellulose fibers.

The layer absorbent material may include comprise at least 30 wt %, or at least 40 wt %, or at least 50 wt %, or at least 60 wt %, or at least 70 wt %, or at least 80 wt % or at least 90 wt % of superabsorbent polymer, such as superabsorbent polymer particles, by total weight of the layer of absorbent material. The layer of absorbent material may comprise less than 25 wt %, or less than 20 wt %, or less than 15 wt %, or less than 10 wt % of cellulose, or less than 5% by weight of cellulose, or even no cellulose based on the total weight of the layer of absorbent material. Absorbent articles having a high percentage of absorbent material, while it may be typically thin and conformable, may cause users to feel a negative touch feeling known as “graininess”.

The superabsorbent polymer particles and the cellulose fibers may be homogeneously mixed with each other such that the ratio of cellulose fibers to superabsorbent polymer particles is substantially the same throughout the layer of absorbent material. Alternatively, the superabsorbent polymer particles and the cellulose fibers may be non-homogeneously mixed such that the ratio of cellulose fibers to superabsorbent polymer particles is higher towards the front and rear edges of the layer of absorbent material compared to a central area of the layer of absorbent material. The area towards the front edge of the layer of absorbent material, the area towards the rear edge of the layer of absorbent material, and the central area may each extend along 1/3 of longitudinal dimension of the layer of absorbent material along the longitudinal centerline.

When the layer of absorbent material is cellulose free, the only absorbent material in the layer of absorbent material may be superabsorbent polymer (particles, fibers or foam). The resulting layer of absorbent material has a reduced thickness in the dry state compared to conventional absorbent cores including cellulosic fibers. The reduced thickness helps to improve the fit and comfort of the absorbent article for the wearer.

The layer of absorbent material may comprise one or more areas where no absorbent material is present, and which are completely surrounded by absorbent material. Hence, these areas may free of cellulose fibers and superabsorbent polymer particles. The areas being free of absorbent material may be elongated areas having a length of from 20% and 80%, or from 20% to 70%, or from 30% to 60%, by total longitudinal dimension of the layer of absorbent material. The elongate areas may be straight, curved, or combinations thereof. The layer of absorbent material may only have one area free of absorbent material or may comprise two or more areas free of absorbent material. Two or more areas free absorbent material may be configured such that they are symmetric with respect to the longitudinal centerline of the absorbent article.

The superabsorbent polymer particles may be selected from among polyacrylates and polyacrylate based materials that are internally and/or surface cross-linked, such as for example partially neutralized cross-linked polyacrylates or acid polyacrylate. Examples of absorbent polymer particles suitable in the present disclosure are described for instance in the WO 07/047598, WO 07/046052, WO2009/155265 and WO2009/155264.

The layer of absorbent material 28 may further comprise longitudinally-oriented channels which are areas substantially free of absorbent material, that facilitate the distribution of a fluid along the length of the absorbent article. These channels preferably do not extend to any of the side of the absorbent layer, and thus the channels are completely surrounded by the absorbent material. The channels are typically elongated in the longitudinal direction, having a length of from 20% and 80%, or from 20% to 70%, or from 30% to 60%, by total longitudinal dimension of the layer of absorbent material 28. The channel may be straight, curved, or combinations thereof. The channels are typically symmetrically disposed relative to the longitudinal axis, and may be disconnected from another, alternatively the channels may be connected at one or both their extremities to form a U or O shape. Such channels are disclosed in further details e.g. in WO2012170778A1, WO2012170781 (Kreuzer et al.).

The layer of absorbent material may be supported by one or more substrate layers such as an upper substrate layer and a lower substrate layer 46. The upper substrate layer 45 may be provided between the topsheet 24 and the layer of absorbent material 28. The upper substrate layer 45 may be provided between the upper ADS 50 and the layer of absorbent material 28. If the absorbent article does not comprise an upper ADS, the upper substrate layer may be provided between the topsheet and the layer of absorbent material. A lower substrate layer 46 may be provided between the layer of absorbent material 28 and the intermediate layer 60. Alternatively to the lower substrate layer 46, the intermediate layer 60 may be in direct contact with the layer of absorbent material 28 (i.e. there is no lower substrate layer 46).

The upper substrate layer 45 and the lower substrate layer 46 may partly or fully enclose the layer of absorbent material 28. Alternatively, the upper substrate layer 45 and the intermediate layer 60 may partly or fully enclose the layer of absorbent material 28.

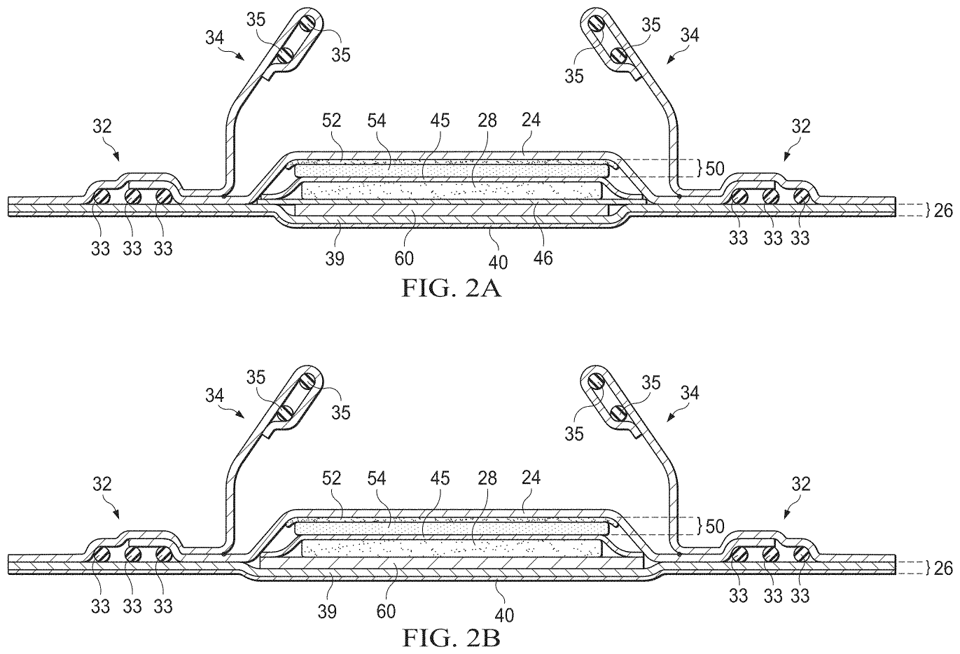

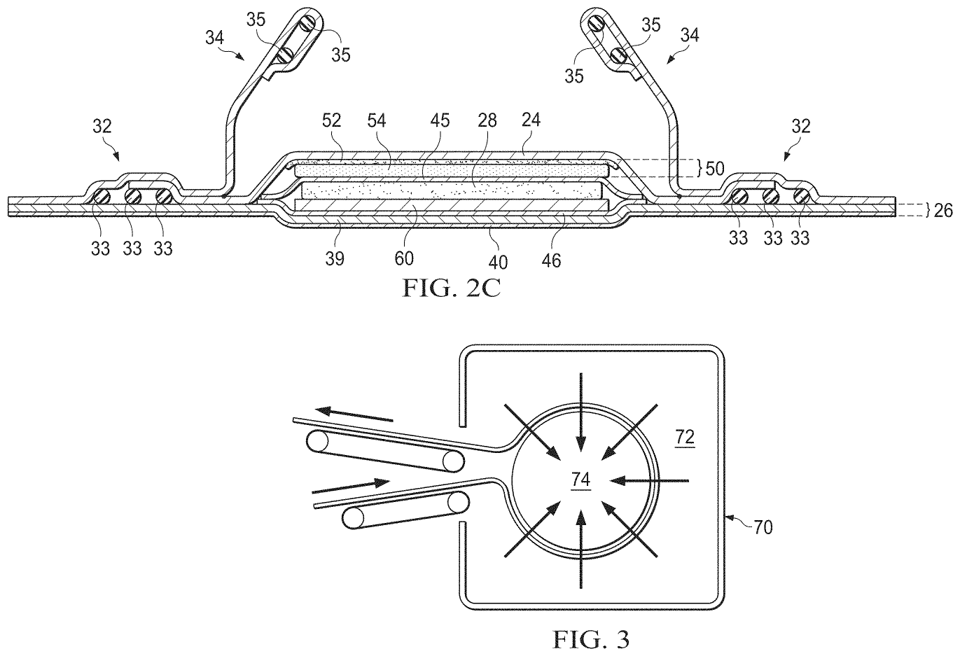

The layer of absorbent material 28 may be partly or fully enclosed by and in direct contact with a substrate layer such as an upper substrate layer 45 generally contacting a top side of the layer of absorbent material 28, and a lower substrate layer 46 generally contacting a bottom side of the layer of absorbent material 28. The upper and lower substrate layers 45 and 46 may be formed by a single substrate, or may be formed two separate substrates which may be the same or different material. The upper substrate layer 45 may be placed facing towards the topsheet 24, that is, the upper substrate layer 45 between the topsheet 24 and the layer of absorbent material 28 when the absorbent article does not include the upper acquisition and distribution system 50, or between the upper acquisition and distribution system 50 and the layer of absorbent material 28 when the absorbent article includes the upper acquisition and distribution system 50. The lower substrate layer 46 may be between the layer of absorbent material 28 and the intermediate layer 60. Such an embodiment is exemplarily shown in FIG. 2A. In such embodiments, a) the intermediate layer 60 may be hydrophobic and the lower substrate layer 46 may be hydrophilic; or b) the intermediate layer 60 and the lower substrate layer 46 may both be hydrophilic and the intermediate layer 60 may be less hydrophilic than the lower substrate layer 46; or c) the intermediate layer 60 and the lower substrate layer 46 may both be hydrophobic and the lower substrate layer 46 may be less hydrophobic than the intermediate layer 60.

Alternatively, the layer of absorbent material 28 and the intermediate layer 60 may be partly or fully enclosed by and in direct contact with an upper and a lower substrate layer 45, 46, and the upper substrate layer 45 may face toward the topsheet 24, and the lower substrate layer 46 may be between the intermediate layer 60 and the backsheet, with the layer of absorbent material 28 being in direct contact with the intermediate layer 60. An embodiment of such configuration is exemplified in FIG. 2C.

In still another alternative, the layer of absorbent material 28 may be partly or fully enclosed by and in direct contact with an upper substrate layer 45 and the intermediate layer 60, and the upper substrate layer 45 may face toward the topsheet 24. Such an embodiment is exemplarily illustrated in FIG. 2B.

Portions at and adjacent to the longitudinal edges of the upper substrate layer 45 may be folded over the longitudinal edges of the layer of absorbent material, such that these portions are positioned on the garment-facing surface of the layer of absorbent material. Alternatively or in addition, portions at and adjacent to the longitudinal edges of the lower substrate layer 46 may be folded over the longitudinal edges of the layer of absorbent material, such that these portions are positioned on the body-facing surface of the layer of absorbent material The layer of absorbent material may be immobilized on the upper substrate layer 45 and/or on the lower substrate layer 46, and/or on the intermediate layer 60, for example by use of hot melt adhesive.

The presence of the intermediate layer according to the present disclosure having high thickness/basis weight under compression and high air permeability as reflected by various examples below can create significant void volume in the intermediate layer, and the intermediate layer may improve the fluid handling performance of the absorbent article of the present disclosure.

Due to the presence of the intermediate layer, the absorbent article of the present disclosure has fast acquisition speed, as is reflected by the acquisition time of the various examples below.

The absorbent article of the present disclosure may comprise an upper ADS 50 disposed between the layer of absorbent material 28 and the topsheet 24. The upper ADS may be in direct contact with the layer of absorbent material 28 and with the topsheet 24. If the absorbent article comprises an upper substrate layer that at least partially encloses the layer of absorbent material 28, the upper ADS may be in direct contact with the topsheet 24 and the upper substrate layer 45.

The upper ADS may serve as a temporary reservoir for liquid until the layer of absorbent material can absorb and store the liquid, and for subsequent distribution of the liquid into the layer of absorbent material in an efficient manner The upper ADS may consist of a single layer or comprise multiple layers, such as an upper layer 52 provided adjacent to the topsheet 24 and facing towards the wearer's body, and a lower layer 54 provided between the upper layer 52 and the layer of absorbent material, facing towards garment of the wearer.

The upper ADS may be free of superabsorbent polymer. The upper ADS may also be free of unmodified cellulose fibers.

The function of a lower layer 54 of the upper ADS 50 is typically to spread the insulting fluid liquid over a larger surface within the absorbent article so that the absorbent capacity of the layer of absorbent material can be more efficiently used. The upper ADS may further comprise an upper layer 52, whose function is typically to quickly acquire the fluid away from the topsheet so as to provide a good dryness for the wearer.

Intermediate Layer

The absorbent article of the present disclosure comprises an intermediate layer between the layer of absorbent material and the backsheet. The intermediate layer 60 may be in direct contact with the layer of absorbent material 28 and with the backsheet 26. If the absorbent article comprises a lower substrate layer 46 that, in conjunction with an upper substrate layer 45 at least partially encloses the layer of absorbent material 28, the intermediate layer 60 may be in direct contact with the topsheet 24 and the lower substrate layer 46.

The intermediate layer may be useful as a masking layer to isolate the superabsorbent polymer particles in the layer of absorbent material from the backsheet, thus reducing graininess feeling and improving the tactile properties of the garment-facing side of the article, especially for absorbent article containing a high level of superabsorbent polymer particles.

The intermediate layer may further isolate the exudates which have been absorbed in the layer of absorbent material from the garment-facing side of the article, as this may be visually unpleasant to the caregiver. Thus by having an intermediate layer with a relatively high opacity, stains in the layer of absorbent material (e.g. from urine or feces) can be concealed from view, when looking at the backsheet of the absorbent article during use. The dry opacity of the intermediate layer may be at least 25%, or at least 40%, or at least 50%, or at least 70%. The intermediate layer can also help reducing the residual moisture in contact with the backsheet, which lead to cold/wet feeling for the caregiver and the wearer sometime mistakenly taken as liquid leaking out of the absorbent article.

The intermediate layer may also be useful to be a temporary reservoir for liquid that has flown through the layer of absorbent material because it was not absorbed fast enough by the absorbent material of the layer of absorbent material.

Additional layers provided to an absorbent article generally increase the thickness and bulkiness of the article. This may lead to increased bending stiffness including stiffness in crotch, and thus to drawbacks for conformity and close contact of the article to the wearer's body, thereby reducing wearer comfort. Also, increased bulk is generally not desirable, especially between the wearer's legs. Therefore, employment of an additional layer in an absorbent article to mitigate grainy feel tends to compromise cushiony feel and/or flexibility of the absorbent article.

Therefore, it is desirable for an intermediate material to have a thickness that can survive compressive force with a minimum possible stiffness. At the same time the intermediate layer is wanted to have a cushiony benefit under compression conditions demonstrated by compression average rigidity given raw material nonwoven is transported and stored wound in rolls and absorbent articles are marketed in a compressed package bag.

The intermediate layer of the present disclosure can provide a high thickness under pressure that can be preserved under relevant compressive forces as reflected by high thickness/basis weight as measured FTT Test so that it can effectively reduce or mask a grainy feel. Yet it is easy to deform or compress as reflected by reflected by compression average rigidity (“CAR”) as measured according to FTT Test so that it can deliver desirable cushiony feel. At the same time, the intermediate layer is flexible as reflected by low bending work as measured according to FTT Test so that it does not to create uncomfortable crotch stiffness.

The intermediate layer having a high thickness/basis weight may effectively reduce an undesirable grainy feel without unnecessary increase of basis weight of the intermediate layer, and provide sufficient void volume to be able to acquire and hold fluid that penetrates through the layer of absorbent material.

The intermediate layer disclosed herein, to not unduly increase the stiffness of the absorbent article, has a MD tensile/basis weight no greater than about 0.75 N/5 cm/g/m², or about 0.71 N/5 cm/g/m² as measured according to Tensile Strength Test disclosed herein. Lower values for MD tensile/g/m² indicate that a material is less bonded and more flexible versus higher values. The intermediate layer has a thickness/basis weight no less than about 0.078 mm/g/m², or about 0.80 mm/g/m², or about 0.90 mm/g/m² as measured FTT Test disclosed herein. Higher values for thickness/basis weight indicate that a material is loftier under compression versus lower values.

The intermediate layer may have bending work no greater than about 2000 gf×mm×rad, or about 1500 gf×mm×rad, or about 1300 gf×mm×rad, as measured according to FTT Test. Lower values for bending work indicate that a material is less stiff versus higher values.

The intermediate layer may have a compression average rigidity no greater than about 400 gf/mm³, or no greater than about 350 gf/mm³, or no greater than about 330 gf/mm³, as measured according to FTT Test. Lower values for compression (or recovery) average rigidity indicate that a material is more cushiony versus higher values.

The basis weight of the intermediate layer may be homogeneous throughout the length and width of the intermediate layer (i.e. in the longitudinal and transverse direction). Such homogeneous basis weight should not take into account local basis weight variations on a rather small scale (such as variations within 1.0 cm, or within 0.5 cm in width and length direction), which may result from mechanical deformation of the intermediate layer.

The intermediate layer may have a smaller extension in the longitudinal and/or transverse direction than the layer of absorbent material, such that the layer of absorbent material extends beyond the intermediate layer in longitudinal and/or transverse direction. The layer of absorbent material may also extend beyond the upper ADS in the longitudinal and/or transverse direction.

Alternatively, the intermediate layer may have a larger extension in the longitudinal and/or transverse direction than the layer of absorbent material, such that the intermediate layer extends beyond the layer of absorbent material in the longitudinal and/or transverse direction. This may be desirable when the layer of absorbent material is in direct contact with the intermediate layer (i.e. when there is no lower substrate layer between the layer of absorbent material and the intermediate layer). In such configurations, the layer of absorbent material may be partly or fully deposited and formed on the intermediate layer. The layer of absorbent material may be partly formed on the intermediate layer and partly on an upper substrate layer, and subsequently, both sub-components of the layer of absorbent material are combined to form the layer of absorbent material by putting the two sub-components in a face to face relationship.

The intermediate layer may be free of superabsorbent polymer. The intermediate layer may comprise or consist of a nonwoven web. It may be a carded air-through bonded nonwoven, a carded calendar bonded nonwovens nonwoven web, a spunbond or meltblown nonwoven web (made of continuous fibers) or a nonwoven with spunbond and meltblown layers (e.g. an SMS, SMMS, SMSS or the like). In one embodiment, the intermediate layer is a carded air-through bonded nonwoven. The nonwoven web may be made of synthetic fibers, such as polyolefin (e.g. polyethylene, polypropylene or mixtures or combinations thereof), polyethylene terephthalate (PET), co-PET, polylactic acid (PLA), polyhydroxy alkanoid (PHA), or combinations or mixtures thereof. The fibers may be continuous or staple fibers.

The intermediate layer may comprise a nonwoven web comprising first thermoplastic fibers having a first melting temperature and second thermoplastic fibers having a second melting temperature, a difference of the first melting temperature and the second melting temperature is at least about 40° C., or at least 50° C., or at least 60° C. If melting temperature of different fiber types get close more or all fiber types will bond to each other and/or to itself which will result in excessive stiffness which is not desired. When the first thermoplastic fiber comprises at least two polymers having different melting temperatures, a melting temperature of a polymer lower than melting temperature(s) of any other polymer(s) constituting the first thermoplastic fiber is considered the first melting temperature. By the same token, when the second thermoplastic fiber comprises at least two polymers having different melting temperatures, one melting temperature of a polymer lower than melting temperature(s) of any other polymer(s) constituting the second thermoplastic fiber is considered the second melting temperature.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 40 wt %, or at least 50 wt %, or at least 60 wt % of the first or the second thermoplastic fibers whichever having a lower melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., the nonwoven web may comprise at least 30 wt %, or at least 40 wt %, or at least 50 wt % of the first or the second thermoplastic fibers whichever having a higher melting temperature based on the total weight of the nonwoven web.

In the embodiment where the nonwoven web comprised by or forming the intermediate layer comprises first thermoplastic fibers having a first melting temperature and second thermoplastic fibers having a second melting temperature, a difference of the first melting temperature and the second melting temperature is at least about 40° C., fibers having a lower melting temperature may be heat-fused one another, and/or substantial part of fibers having a higher melting temperature may not heat-fused one another.

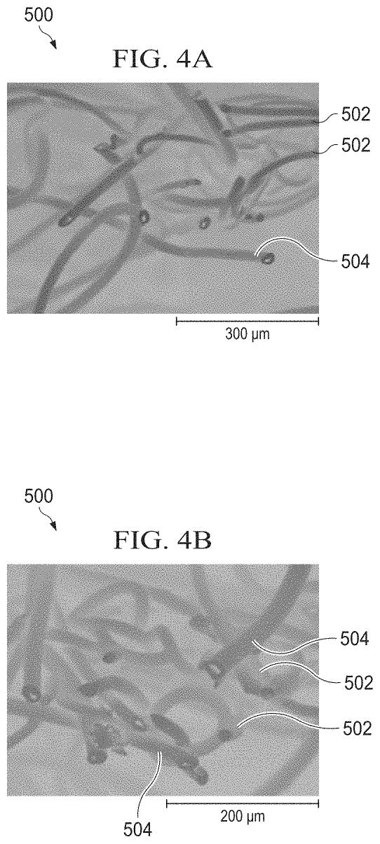

In one embodiment when the second thermoplastic fibers have a melting temperature greater at least about 40° C. than the first thermoplastic fibers, referring to FIGS. 4A and 4B which are SEM images of a cross-sectional view of Nonwoven 1, the first thermoplastic fibers 502 having a melting temperature lower than the second thermoplastic fibers 504, hollow fibers in this case, in the nonwoven web 500 are heat-fused one another. The presence of the first thermoplastic fibers which are not heat-fused one another is acceptable as long as majority of the first thermoplastic fibers are heat-fused one another. Still referring to FIGS. 4A and 4B, the second thermoplastic fibers 504, hollow fibers in this case, in the nonwoven web 500 are not heat-fused one another. Further, majorities of the first thermoplastic fibers and the second thermoplastic fibers may not be heat-fused each another.

Without being bound by theory, optimizing fiber to fiber bonding per mass of nonwoven web may enable the intermediate layer to have a high thickness under compression and a low stiffness especially in the crotch. Increase of fiber to fiber bonding in the nonwoven may increase the stiffness of the material. On the other hand, decrease of fiber-to-fiber bonding in nonwoven web may result in less integrity of the nonwoven which is more prone to collapse of the material under compressive forces.

The first thermoplastic fiber may be a solid round fiber, a hollow fiber or a shaped fiber. The second thermoplastic fiber may be a solid round fiber, a hollow fiber or a shaped fiber. In one embodiment, the second thermoplastic fiber is a hollow fiber or a shaped fiber. In the embodiment, the second thermoplastic fiber may be hollow conjugate fiber.

Shapes fibers also may introduce higher specific surface area which increases the capillary pressure of the second web layer containing shaped fibers which can lead to better drainage of the first web layer by the second fiber web layer comprising shape fibers. Shaped fibers may include bilobal shaped, trilobal shaped, quatro-lobal shaped, delta shaped, concave delta shaped, crescent shaped, oval shaped, star shaped, square shaped, U-shaped, H-shaped, C-shaped, V-shaped, diamond shaped fibers.

Hollow fibers enable greater loft with larger effective diameter per linear density with less weight. They also provide better resilience under compression. Hallow fibers can be hollow conjugate fibers with spiral and/or 3D crimp to maximize the benefits of loft and resilience. Such hollow conjugate fibers can have non-uniform properties across the fiber cross-section for instance by using polymers with different characteristics (e.g. different polymers or same polymer with different characteristics such as viscosity).

Without being bound by theory, hollow fibers or shaped fibers may be advantageous over solid round fibers to provide improved cushiony characteristics as hollow fibers and shaped fibers have higher resilience at the same fiber denier due to having higher effective radius compared to round fibers.

Each of the first and the second thermoplastic fibers may be monocomponent fibers or multicomponent fibers, such as bicomponent fibers. If the fibers are bicomponent fibers, they have a core-sheath configuration, wherein the core component has a higher melting temperature than the sheath component.

The intermediate layer comprises or consists of a nonwoven web which is air-through bonded. Such nonwoven webs generally have high loft. Hence, they have a porous structure to provide void volume for absorbing and temporarily holding liquid. At the same time, they provide softness and do not have an excessively high bending stiffness.

The fibers may be continuous, such as in a spunlaid nonwoven web. The spunlaid nonwoven web is preferably air-through bonded or spunlace. In addition to hydroentanglement (spunlace) or air-through bonding, the spunlaid nonwoven web may or may not have undergone some localized bonding with heat and/or pressure (e.g. point bonding), introducing localized bond regions where the fibers are fused to each other.

In some embodiments, the fibers comprised by the intermediate layer are staple fibers. Similar to a nonwoven web made of continuous fibers, a nonwoven web of staple fibers is preferably carded nonwoven such as air-through bonding nonwoven. In addition to air-through bonding, the nonwoven web of staple fibers may or may not have undergone some localized bonding with heat and/or pressure (e.g. point bonding), introducing localized bond regions where the fibers are fused to each other.

Irrespective whether the nonwoven web is made of continuous fibers or staple fibers, the localized bonding should however not bond an excessively large surface area, thus negatively impacting the loft and void volume of the nonwoven web as well as stiffness. Preferably, the total bond area obtained by localized bonding with heat and/or pressure (in addition to hydroentanglement or air-through bonding) should not be more than 20%, or not be more than 15%, or not be more than 10% of the total surface area of the nonwoven web.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

Alternatively, the nonwoven web comprised by the intermediate layer should not have undergone any bonding and consolidation in addition to the hydroentanglement (spunlace) or air-through bonding. Thereby, the advantageous properties of such nonwoven webs can be used to their optimum.

In a spunlace nonwoven web the fibers have been subjected to hydroentanglement to intermingle and intertwine the fibers with each other. Cohesion and the interlacing of the fibers with one another may be obtained by means of a plurality of jets of water under pressure passing through a moving fleece or cloth and, like needles, causing the fibers to intermingle with one another. Thus, consolidation of a spunlace nonwoven web is essentially a result of hydraulic interlacing. “Spunlace nonwoven web”, as used herein, also relates to a nonwoven formed of two or more precursor webs, which are combined with each other by hydraulic interlacing. The two or more webs, prior to being combined into one nonwoven by hydraulic interlacing, may have underdone bonding processes, such as heat and/or pressure bonding by using e.g. a patterned calendar roll and an anvil roll to impart a bonding pattern. However, the two or more webs are combined with each other solely by hydraulic interlacing. Alternatively, the spunlace nonwoven web is a single web, i.e. it is not formed of two or more precursor webs. Spunlace nonwoven layers/webs can be made of staple fibers or continuous fibers.

Through-air bonding (interchangeably used with the term “air-through bonding”) means a process of bonding staple fibers or continuous fibers by forcing air through the nonwoven web, wherein the air is sufficiently hot to melt (or at least partly melt, or melt to a state where the fiber surface becomes sufficiently tacky) the polymer of a fiber or, if the fibers are multicomponent fibers, wherein the air is sufficiently hot to melt (or at least partly melt, or melt to a state where the fiber surface becomes sufficiently tacky) one of the polymers of which the fibers of the nonwoven web are made. The air velocity is typically between 30 and 90 meter per minute and the dwell time may be as long as 6 seconds. The melting and re-solidification of the polymer provide the bonding between different fibers.

A through air bonder is schematically shown in FIG. 3. In the through-air bonder 70, air having a temperature above the melting temperature of the polymer of the staple fibers or continuous fibers or, if the staple or continuous fibers are multicomponent fibers, above the melting temperature of a first fiber component and below the melting temperature of a second fiber component, is directed from the hood 72, through the web, and into the perforated roller 74. Alternatively, the through-air bonder may be a flat arrangement wherein the air is directed vertically downward onto the web. The operating conditions of the two configurations are similar, the primary difference being the geometry of the web during bonding.

The hot air melts the staple or continuous fiber, or, for multicomponent fibers, the lower melting polymer component of the fiber and thereby forms bonds between the staple fibers to consolidate and integrate the layer of staple fibers into a web.

In one embodiment, the nonwoven useful for the intermediate layer may be produced by a process comprising supplying a nonwoven web comprising first thermoplastic fibers having a first melting temperature and second thermoplastic fibers having a second melting temperature, wherein the second melting temperature is at least about 40° C. higher than the first melting temperature and, applying heat to the nonwoven web so that at least part of the first thermoplastic fibers are heat-fused one another, wherein the heat has a temperature between the first melting temperature and lower than the second melting temperature.

Measurement of melting temperatures of polymers, and melting temperatures of polymer components of fibers forming nonwoven have been well known in many references such as Differential Scanning Calorimetry of Polymer (Ellis Horwood, 1994) and Handbook of Nonwovens (Elsevier Science, 2007). Melting temperatures of some polymers widely used in fibers forming nonwoven, referred Handbook of Nonwovens (Elsevier Science, 2007), are as below.

CoPET: 100-110° C.

PET: 245-265° C.

PP: 160-175° C.

PE (low density): 115° C.

PE (high density): 125-135° C.

The nonwoven comprised by or forming the intermediate layer may comprise multicomponent fibers. The fibers of the nonwoven comprised by the intermediate layer, may comprise at least 30 wt %, or at least 40 wt %, or at least 50 wt %, or at least 70 wt %, or at least 90 wt % of multicomponent fibers based on the total weight of the nonwoven comprised by the intermediate layer. The nonwoven web may comprise multicomponent fibers no greater than 90 wt %, or no greater than 80 wt %, or no greater than 70 wt % of multicomponent fibers based on the total weight of the nonwoven web comprised by the intermediate layer.

Additionally, or alternatively, the nonwoven comprised by or forming the intermediate layer may comprise monocomponent fibers. The fibers of the nonwoven web comprised by the intermediate layer, may comprise at least at least 20 wt %, or at least 30 wt %, or at least 40 wt % of monocomponent fibers based on the total weight of the nonwoven web comprised by the intermediate layer. The nonwoven web may comprise monocomponent fibers no greater than 70 wt %, or no greater than 60 wt %, or no greater 50 wt % of monocomponent fibers based on the total weight of the nonwoven web comprised by the intermediate layer.

The nonwoven web comprised by or forming the intermediate layer may comprise a mixture of monocomponent fibers and multicomponent fibers. The fibers of the nonwoven web comprised by the intermediate layer, may comprise at least 20 wt %, or at least 30 wt %, or at least 40 w % of monocomponent fibers based on the total weight of the nonwoven web comprised by the intermediate layer.

The nonwoven web comprised by or forming the intermediate layer may comprise third fibers in addition to the first and second thermoplastic fibers. The third fibers may not be heat-fused with the first thermoplastic fibers and second thermoplastic fibers. Examples of the third fibers include synthetic fibers such as acrylic-based, polyester-based, polyamide-based, polyolefin-based, and polyurethane-based fibers; natural fibers such as cotton, silk, wool, hemp, pulp, and the like; and reclaimed fiber such as rayon, cupra, and the like.

As the nonwoven layer of the intermediate layer is preferably a very lofty structure, the use of crimped fibers may be beneficial. Such fibers have also shown to provide the nonwoven layer with good resiliency, i.e. the nonwoven web has a relatively good ability to regain its original thickness (or most of its original thickness) after it has been compressed for a longer time (e.g. while being contained in a closed package that contains highly compressed absorbent articles). The crimped fibers may have flat crimp (so-called two-dimensional crimp) or three-dimensional crimp, such as spiral crimp. Bicomponent fibers are well known as being suitable for obtaining crimped fibers.

The fibers of the nonwoven comprised by the intermediate layer, may comprise at least 30 wt %, or at least 40 wt %, or at least 50 wt %, or at least 70 wt %, or at least 90 wt % or 100 wt % of crimped fibers based on the total weight of the nonwoven comprised by the intermediate layer. The crimped fibers may be bicomponent fibers.

The basis weight of the intermediate layer may be from 20 g/m² to 100 g/m², or from 20 g/m² to 80 g/m², or from 20 g/m² to 60 g/m².

The nonwoven comprised by or forming the intermediate layer may have undergone mechanical deformation. Such mechanical deformation can contribute to the loft and openness of the nonwoven web, hence improving those properties of the nonwoven web which are desirable for use as intermediate layer.

If the wearer- and/or garment facing surfaces of the intermediate layer have a three-dimensional surface topography (as may, for example, be obtained by mechanical deformation), so-called “air pockets” may be obtained, especially if the intermediate layer is in direct contact with layer (such as backsheet or lower substrate layer 46) having flat, two-dimensional surface topography. The three-dimensional surface of the intermediate layer may only contact the adjacent layer (such as the backsheet or lower substrate layer 46) in areas protruding outwardly, leaving small gaps in the areas which are recessed. These gaps can increase wearer-comfort and soft feel of the absorbent article.

Also, it is desirable to have good air permeability of the intermediate layer. As adding an intermediate layer means adding an additional layer of material to the absorbent article, such additional layer should not excessively impact the overall air permeability of the absorbent article. By using a porous, relatively open structure of the nonwoven web of the intermediate layer, such as a spunlace or air-through bonded nonwoven, suitable air permeability of the intermediate layer can be obtained.

The intermediate layer 60 may have an air permeability greater than 150 m³/m²/min, or from 200 m³/m²/min to 800 m³/m²/min, as determined by the test method set out below.

Bio-Sourced Materials

Components of the disposable absorbent article (i.e., diaper, pant, sanitary napkin, pantiliner, etc.) of the present disclosure can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, barrier leg cuff nonwovens, superabsorbent material, upper and lower substrate layer, adhesives, fastener hooks, and fastener landing zone nonwovens and film based. For example, the upper and/or intermediate layer of the present disclosure may at least partially be comprised of bio-sourced content.

The disposable absorbent article component may comprise a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. Thereto, the disposable absorbent article component may be ground into particulates less than about 20 mesh using known grinding methods (e.g., Wiley® mill), and a representative sample of suitable mass taken from the randomly mixed particles.

Packages

A plurality of articles according to the present disclosure may be packaged in a package for transport and sale. At least 50% of the articles, and preferably all the articles, in the package may be according to the present disclosure. The articles may be folded and packaged as is known in the art. The package may be for example a plastic bag or a cardboard box. Diapers may typically bi-folded along the transversal axis and the ears folded inwardly before being packaged. The absorbent articles may be packed under compression so as to reduce the size of the packages, while still providing an adequate amount of absorbent articles per package. By packaging the absorbent articles under compression, caregivers can easily handle and store the packages, while also providing distribution and inventory savings to manufacturers owing to the size of the packages.

The absorbent articles may thus be packaged compressed at an In-Bag Compression Rate of at least 10%, in particular of from 10% to 50%, in particular from 20% to 40%. The “In-Bag Compression Rate” as used herein is one minus the height of a stack of 10 folded articles measured while under compression within a bag (“In-Bag Stack Height”) divided by the height of a stack of 10 folded articles of the same type before compression, multiplied by 100; i.e. (1-In-Bag Stack Height/stack height before compression)*100, reported as a percentage. Of course, the stack in the bag does not need to have exactly 10 articles, rather the value measured for the height of stack of article in the package is divided by the number of articles in the stack and then multiplied by 10. The method used to measure the In-Bag Stack Height is described in further details in the Test Procedures. The articles before compression are sampled from the production line between the folding unit and the stack packing unit. The stack height before compression is measured by taking 10 articles before compression and packing, and measuring their stack height as indicated for the IBSH.

Measurement

1. Tensile Strength Test

MD tensile strength of a specimen is measured according to NWSP 110.4-09 with conditions below.
Test Speed: 100 mm/min
Sample Width: 50 mm
Sample length: sufficiently longer than gauge length
Gauge Length: 100 mm

2. Fabric Touch Tester Test (FTT Test)

The Compression Average Rigidity (CAR), Standard Thickness (T), and Bending Work (BW) values are measured on a nonwoven test sample using a Fabric Touch Tester M293 (FTT), available from SDL Atlas USA, Rock Hill, S.C., interfaced with a computer running FTT system software. According to SDL Atlas, the FTT objectively and quantitatively characterizes skin touch comfort by measuring various mechanical and surface properties. The FTT instrument offers a variety of assessment modules to measure these properties. The FTT Test utilizes the Compression Module, which compresses a sample between two plates while recording the applied normal force and corresponding distance between the plates during a compression and recovery cycle. The FTT Test also utilizes the Bending Module, which bends a sample over a bending bar while recording the bending force and corresponding bending angle. The recorded data is analyzed by the FTT software to calculate the CAR, T, and BW values. The instrument operation and testing procedures are performed according to the instrument manufacture's specifications.

2.1 Sample Preparation

When a nonwoven is available in a raw material form, a rectangular test sample with a size of 310 mm×90 mm is cut from the raw material. When a nonwoven is a component of a finished product, the nonwoven is removed from the finished product using a razor blade to excise the nonwoven from other components of the finished product to provide a nonwoven test sample with a size of 310 mm×110 mm A cryogenic spray (such as Cyto-Freeze, Control Company, Houston Tex.) may be used to remove the nonwoven specimen from other components of the finished product, if necessary. Equilibrate all samples at TAPPI standard temperature and relative humidity conditions (23° C.±2° C. and 50%±2%) for at least 4 hours prior to conducting the FTT testing, which is also conducted under TAPPI conditions.

2.2 Testing Procedure

The FTT instrument is calibrated according to the manufacturer's instructions using the provided standard calibration fabric. The test sample is placed into the instrument according to the manufacturer's instructions, with the appropriate amount of the sample laying on the compression plate and the remaining portion resting on the adjacent bending platform. The test sample should be laying flat and tension free prior to initiating the test. The compression and bending tests are initiated and performed according to the manufacturer's instructions.

When testing is complete, the FTT software displays values for CAR, T, and BW. Record each of these values. The test piece is then removed from the instrument and discarded. This testing procedure is performed individually on the other four replicate test samples.

The arithmetic means of the five recorded test result values for CAR, T, and BW are calculated and reported. Report the individual average values of CAR to the nearest 1 gf/mm³, T to the nearest 0.01 mm, and BW to the nearest 1 gf·mm·rad.

3. Air Permeability Test

All measurements are performed in a laboratory maintained at 23±2° C. and 50±2% relative humidity and test specimens are conditioned in this environment for at least 2 hours prior to testing.

The Air Permeability of a substrate is determined according to INDA/EDANA Nonwovens Standard Procedures NWSP 070.1.R0 (15) making use of a Textest FX3300 (Textest Instruments, Schwerzenbach, Switzerland) air permeability tester or equivalent. A circular test head with an area of 20 cm² is used, and while a fixed pressure of 200 Pa is maintained across the specimen, air flow through the specimen is measured in cubic meter per square meter per minute (m³/m²/min). If possible, measurements are made on the material before it is integrated in an absorbent article. If this is not possible, care should be exerted when excising the specimen from the product not to impart any contamination or distortion to the test sample layer during the removal of the material from other layers (using cryogenic spray, such as Cyto-Freeze, Control Company, Houston, Tex., if needed). Five rectangular specimens of the material are taken such that each specimen center corresponds to the center of the material and such that the length and width of each specimen are greater than the smallest dimension of the circular head. The specimen is placed under the test head such that the center of the specimen is matching the center of the test head. The five lower specimens are analyzed in this way, and the air permeability of each is recorded in m³/m²/min to the nearest 1 m³/m²/min. The arithmetic mean of the individual specimen results is calculated and reported as the Air Permeability in units m³/m²/min to the nearest 1 m³/m²/min.

4. SEM Image Test

4.1 Sample preparation

When a nonwoven is available in a raw material form, a specimen with a size of 10 mm×10 mm is cut from the raw material. When a nonwoven is a component of a finished product, the nonwoven is removed from the finished product using a razor blade to excise the nonwoven from other components of the finished product and cut to provide a nonwoven specimen with a size of 10 mm×10 mm and free from folds or wrinkles. A cryogenic spray (such as Cyto-Freeze, Control Company, Houston, Tex.) may be used to remove the nonwoven sample from other components of the finished product, if necessary.

For the measurement of a top view of a specimen, the specimen is adhered on a copper plate (25 mm diameter, 20 mm thickness) horizontally by double-sided conductive tape.

For the measurement of a cross section of a specimen, the specimen is firstly soaked in liquid nitrogen for 180 sec and then cut by a steel knife perpendicular to the specimen planar direction. After cutting, the specimen is adhered on a copper plate vertically by double-sided conductive tape, with the cut side facing up.

Then, the plate is placed in a sample chamber of a coating instrument (such as Hitachi E-1045) for platinum-spray coating. During coating, an air pressure in the sample camber is controlled to be lower than 100 Pa, and a charge currency is 30 mA. After coating for 120 sec, the copper plate is taken out.

4.2 Image Generation

The coated specimen adhered on the copper plate is placed in the camber of SEM instrument (Hitachi TM3000) for measurement. For a top view image, an SEM image is obtained at a resolution sufficient to clearly elucidate absorbent fibers and ultrafine fibers present in the specimen. For a cross-section view image, an SEM image is obtained at a resolution sufficient to clearly elucidate the cross sections of the fibers present in the specimen.

5. Modified Fluid Acquisition Test

The Modified Fluid Acquisition (“MFA”) Test is designed to measure the speed at which 0.9% saline solution is absorbed into an absorbent article that is compressed at 2.07 kPa. A known volume is introduced four times, each successive dose starting five (5) minutes after the previous dose has absorbed. Times needed to absorb each dose are recorded. The test fluid is 0.9% w/v saline solution and is prepared by weighing 9.0 g±0.05 g of NaCl into a weigh boat, transferring it into a 1 L volumetric flask, and diluting to volume with de-ionized water.

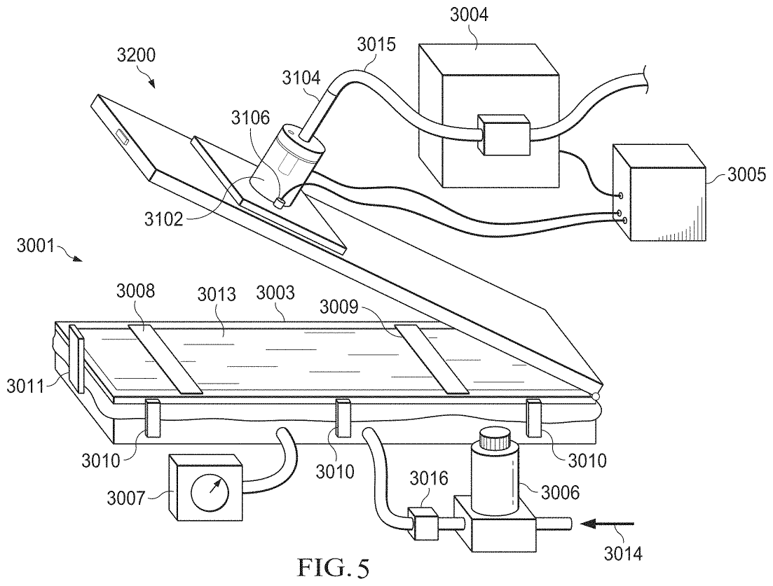

The MFA apparatus is depicted in FIG. 5 through FIG. 7B. The MFA apparatus comprises a bladder assembly 3001 and a top plate assembly 3200 that includes a deposition assembly 3100. A controller 3005 is used to 1) monitor the impedance across electrodes 3106, recording the time interval 0.9% saline solution is in a cylinder 3102, 2) interface with a liquid pump 3004 to start/stop dispensing, and 3) time intervals between dosing. The controller 3005 is capable of recording time events to ±0.01 sec. A house air supply 3014 is connected to a pressure regulator 3006 capable of delivering air at a suitable flow/pressure to maintain 2.07 kPa in the bladder assembly 3001. A liquid pump 3004 (Ismatec MCP-Z gear pump, available from Cole Palmer, Vernon Hills, Ill. or equivalent) capable of delivering a flow of 10-80 mL at a rate of 3-15 mL/s is attached to a steel tube 3104 of the deposition assembly 3100 via tygon tubing 3015.

The bladder assembly 3001 is constructed of 12.7 mm Plexiglas with an overall dimension of 80 cm long by 30 cm wide by 10 cm tall. A manometer 3007 to measure the pressure inside the assembly and a pressure gauge 3006 to regulate the introduction of air into the assembly are installed through two holes through the light side. A bladder 3013 is assembled by draping a 50 mm by 100 mm piece of silicone film, (thickness 0.02″, Shore A durometer value of 20, available as Part#86435K85 from McMaster-Carr, Cleveland, Ohio) over the top of the box with enough slack that the film touches the bottom of the box at its center point. An aluminum frame 3003 with a flange is fitted over the top of the film and secured in place using mechanical clamps 3010. When in place, the assembly should be leak free at a pressure of 3.45 kPa. A front 3008 and back 3009 sample support of 5 cm by 30 cm by 1 mm are used to anchor the sample. The absorbent article is attached to the top surface of the sample supports by either adhesive tape or mechanical “hook” fasteners. These supports can be adjusted along the length of the aluminum frame 3003 via a simple pin and hole system to accommodate different size absorbent articles and to correctly align their loading point.

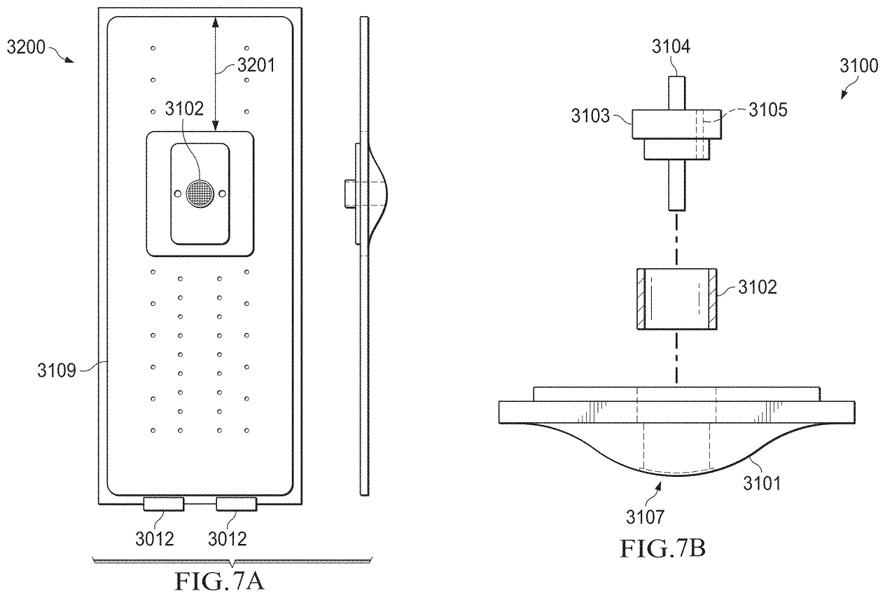

The top plate assembly 3200 is constructed of an 80 cm by 30 cm piece of 12.7 mm Plexiglas reinforced with an aluminum frame 3109 to enhance rigidity. The plate has a cutout 170 mm wide by 201 mm long centered laterally on the plate, 170 mm from the front of the plate 3201 for mounting of the deposition assembly. In addition, the top plate has thirty-six (36) 3.2 mm diameter holes drilled through it distributed as shown in FIG. 7A. The holes prevent air from being trapped under the top plate as the bladder is inflated. The top plate assembly 3200 is connected to the bladder assembly 3001 via two hinges 3012. During use, the top assembly is closed onto the bladder assembly and locked into place using a mechanical clamp 3011.

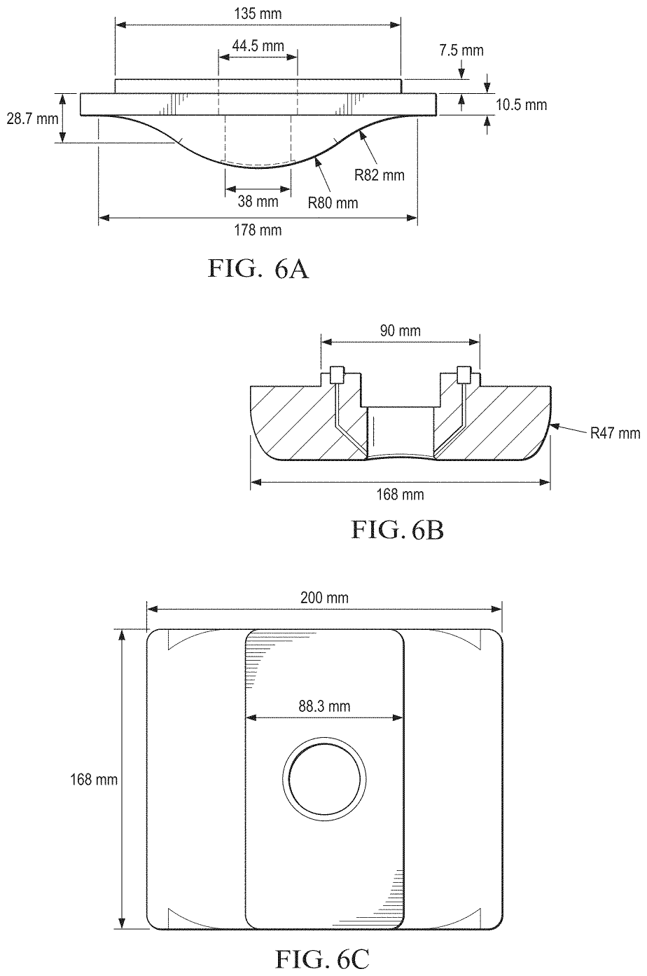

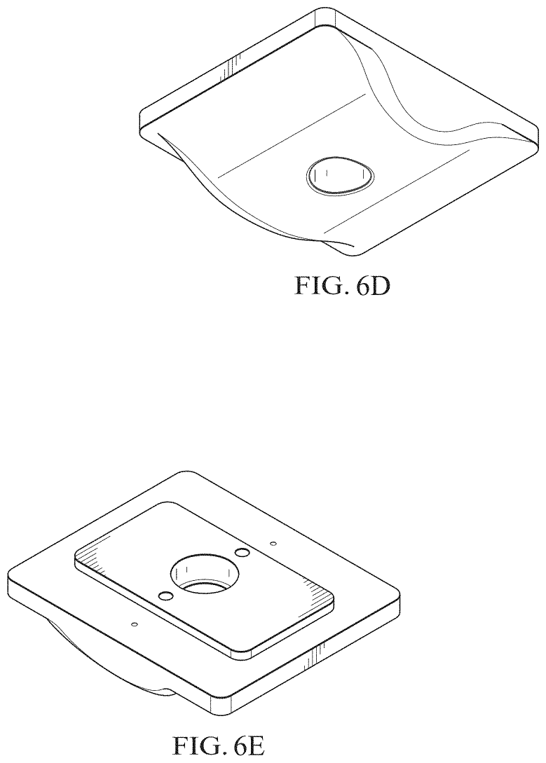

The deposition assembly 3100 is fitted into the top plate 3200 and includes 1) a liquid introduction cylinder 3102, 2) a curved surface 3101 at the loading point of the absorbent article and 3) electrodes 3106 that are used to detect fluid in the cylinder 3102. The detailed dimensions of the curved component are provided in FIG. 6A to FIG. 6E. FIG. 6A is a side view of the curved component. FIG. 6B is an end view of the curved component. FIG. 6C is a bottom view of the curved component. FIG. 6D is a bottom perspective view of the curved component. FIG. 6E is a top perspective view of the curved component. This curved component can be milled or 3D printed. The top portion of the introduction cylinder is a 50.8 mm O.D. Plexiglas cylinder 3102 with a 38.1 mm LD. This is fitted into the curved component to give the introduction cylinder a total height of 100 mm Imbedded electrodes run from connectors on the upper surface of the curved component and terminate flush with an inside wall of the introduction cylinder, 2 mm from the bottom of the cylinder. The two electrodes are positioned 180 degrees apart. A nylon screen 3107 is cut and affixed flush with the bottom of the cylinder such that the sample cannot swell into the cylinder. A 5 mm semi-circle is cut in the screen in the immediate area of the two electrodes. The deposition assembly is inserted into the top plate as shown in FIG. 7A such that the curved surface is flush with the bottom of the top-plate assembly 3200. The introduction cylinder 3102 is topped with a loose-fitting nylon cap 3103. The cap has a 6.35 mm O.D. steel tube 3104 inserted through its center. When the cap is in place, the bottom of the tube ends 20 mm above the screen 3107. The cap also has an air hole 3105 to ensure negative pressure does not impede the absorption speed.

The absorbent article is first prepared by excising any inner or outer leg cuffs, waist caps, elastic ears or side panels, taking care not to disturb the topsheet that resides above the article's core region. Place the absorbent article flat onto a lab bench and identify the intersection of the longitudinal centerline with the size dependent loading point as defined in Table 1.

TABLE 1
Conditions for Modified Fluid Modified Fluid Acquisition Testing:

| | | Loading Point from | | Single | |
|---|---|---|---|---|---|
| | Approximate | front of Core* | | Dose | Flow |
| Diaper | Baby Weight | Boy | Girl | Volume | Rate |
| Size | Pounds | mm | mm | mL | mL/s |
| 1 | 8 to 13 | 64 | 64 | 24 | 8 |
| 2 | 13 to 17 | 76 | 89 | 24 | 8 |
| 3 | 17 to 28 | 89 | 114 | 50 | 10 |
| 4-6 | 28+ | 102 | 127 | 75 | 15 |

*the boy loading point is used for unisex diapers.

Attach the front end of the absorbent article to the top surface of the front sample plate 3008 by either adhesive tape or mechanical “hook” fasteners with a topsheet facing upward. The placement is such that just the chassis and not the absorptive core overlays the plate. The sample plate 3008 is attached to the aluminum frame 3003 such that the size-dependent Loading Point (as defined in Table 1) of the absorbent article will be centered longitudinally and laterally within the cylinder 3102 when the top plate assembly has been closed. The back end of the absorbent article is secured to the back sample plate 3009 by either adhesive tape or mechanical “hook” fasteners, once again ensuring that only the chassis and not the absorptive core overlays the plate. The back sample plate 3009 is then attached to the aluminum frame 3003 such that the article is taunt but not stretched. The top plate assembly is closed and fastened, and the bladder is inflated to 2.07 kPa±0.07 kPa. The pressure is maintained at this level during the complete loading sequence of the test.

The pump 3004 is primed and then calibrated to deliver the size-dependent volume and flow rate selected from Table 1. Volume and flow rate must be within ±2% of target. The cap 3103 is placed into the cylinder 3102. The controller 3005 is started, which in tum delivers the first dose of 0.9% saline solution. After the volume has been absorbed, the controller waits for 5.0 minutes before addition of the next dose. This cycle is repeated for a total of four doses. If the fluid leaks out of or around the article (i.e., is not absorbed into the article) then the test is aborted. Also if any acquisition time exceeds 1200 seconds, the test is aborted. The acquisition time is defined as the difference between the start time (i.e., when the 0.9% saline is first introduced into the cylinder and that conducting fluid completes the circuit between the electrodes) and the stop time (i.e., when the fluid has completely drained from the cylinder and the circuit between the electrodes is broken). Acquisition times are recorded by the controller for each dose to the nearest 0.01 second. After the last dose is acquired, pressure is applied for an additional 10 minutes. Open the pressure relief valve 3016 to deflate the bladder and then remove the sample from the acquisition system.

In like fashion, run a total of sixteen (16) replicates for each absorbent article to be evaluated. Calculate and report the Acquisition Times (sec) for each dose as the arithmetic mean of the replicates to the nearest 0.01 sec.

### EXAMPLES

### Example 1

### Nonwoven Preparation

Various nonwoven sheets specified in Table 2 below were prepared.

TABLE 2

| | Basis | | Manufacturing |
|---|---|---|---|
| Nonwoven | weight (g/m²) | Composition | process |
| Nonwoven 1 | 60 | 60 wt % 2 dpf solid round CoPET/PET | carded air—through |
| | | staple fiber and 40 wt % 3 dpf hallow | bonding |
| | | conjugate PET staple fiber | |
| Nonwoven 2 | 40 | 60 wt % 2 dpf solid round CoPET/PET | carded air—through |
| | | staple fiber and 40 wt % 3 dpf hallow | bonding |
| | | conjugate PET staple fiber | |
| Nonwoven 3 | 40 | 45 wt % 2 dpf trilobal PP staple fiber and | carded calendar |
| | | 55 wt % 6 dpf solid round PET staple fiber | bonding |
| Nonwoven 4 | 60 | 100 wt % 4.0 dpf solid round PE/PET | carded air—through |
| | | staple fiber | bonding |
| Nonwoven 5 | 60 | 60 wt % 2 dpf solid round CoPET/PET | carded air—through |
| | | staple fiber and 40 wt % 3dpf solid round | bonding |
| | | PET staple fiber | |
| Nonwoven 6 | 40 | 60 wt % 2 dpf solid round CoPET/PET | carded air—through |
| | | staple fiber and 40 wt % 3 dpf solid round | bonding |
| | | PET staple fiber | |
| Nonwoven 7 | 40 | 60 wt % 2 dpf solid round CoPET/PET | carded air—through |
| | | staple fiber and 40 wt % 2 dpf solid round | bonding |
| | | PE/PET staple fiber | |

### Example 2

### Nonwoven Properties

Properties of nonwovens manufactured in Example 1 were tested and are displayed in Table 3. MD Tensile was measured according to Tensile Strength Test, and Compression Average Rigidity (CAR), Standard Thickness (T) as thickness, and Bending Work (BW) were measured according to FTT Test. Air permeability was measured according to Air Permeability Test.

Nonwovens 1-3 are embodiments of nonwoven webs of the present disclosure, while nonwovens 4-7 are provided merely for comparison purposes.

TABLE 3

| | Nonwoven 1 | Nonwoven 2 | Nonwoven 3 | Nonwoven 4 | Nonwoven 5 | Nonwoven 6 | Nonwoven 7 |
|---|---|---|---|---|---|---|---|
| Basis weight | 60 | 40 | 40 | 60 | 60 | 40 | 40 |
| (BW, g/m²) | | | | | | | |
| MD Tensile | 42.8 | 23.0 | 16.9 | 118.7 | 47.1 | 31.1 | 40.1 |
| (N/5 cm) | | | | | | | |
| MD Tensile/BW | 0.71 | 0.58 | 0.42 | 1.98 | 0.79 | 0.78 | 1.00 |
| (N/5 cm/g/m²) | | | | | | | |
| Thickness* | 0.623 | 0.435 | 0.331 | 0.420 | 0.453 | 0.340 | 0.253 |
| (T, mm) | | | | | | | |
| Thickness/BW | 0.0104 | 0.0109 | 0.0083 | 0.0070 | 0.0076 | 0.0085 | 0.0063 |
| (mm/g/m²) | | | | | | | |
| Bending Work | 1232 | 767 | 290 | 2246 | 659 | 420 | 490 |
| (gf × mm × rad) | | | | | | | |
| CAR | 220 | 328 | 274 | 124 | 286 | 443 | 571 |
| (gf/mm³) | | | | | | | |
| Air permeability | 234 | 320 | NA | 212 | 221 | 303 | NA |
| (m³/m²/min) | | | | | | | |

Thickness*: The Standard Thickness in the FTT test, when specimen size is a size of 310 mm × 90 mm, measured under pressure of 51 gf/cm².

### Example 3

### Diaper Preparation

Diaper 1-4 as exemplary absorbent articles having intermediate layers were fabricated using a common topsheets, an upper ADS, an upper substrate layer, a layer of absorbent material, a backsheet, and an outer cover nonwoven according to Table 4 below.

TABLE 4

| Diaper | Intermediate layer |
|---|---|
| 1 | Nonwoven 1 |
| 2 | Nonwoven 2 |
| 3 | Nonwoven 4 |
| 4 | Nonwoven 5 |
| 5 | 100% polypropylene SMS |

### Example 4

### Diaper Properties

Acquisition time of Diapers 1-3 and 5 were tested according to Modified Fluid Acquisition Test, and are displayed in Table 5.

TABLE 5

| | | Diaper 1 | Diaper 2 | Diaper 3 | Diaper 5 |
|---|---|---|---|---|---|
| Acquisition time (sec) | 1^{st} gush | 60.4 (1.9*) | 68.2 (2.4*) | 74.6 (2.6*) | 88.0 (2.9*) |
| | 2^{nd} gush | 63.0 (5.1*) | 76.2 (4.3*) | 75.6 (6.9*) | 83.6 (6.2*) |
| | 3^{rd} gush | 119.5 (10.9*) | 137.4 (10.9*) | 164.3 (26.3*) | 150.5 (15.3*) |
| | 4^{th} gush | 336.7 (43.4*) | 369.1 (38.7*) | 822.4 (87.8*) | 471.0 (103.3*) |

*standard deviation

It is important to note that the nonwoven suitable for an intermediate layer in an absorbent article of the present disclosure has a lofted thickness/basis weight under pressure which therefore can provide better masking of a grainy feel and still flexible, and the absorbent articles of the present disclosure also achieves a fast acquisition time in comparison with comparative diapers 3 and 4.

With 4 panelists, stiffness, grainy feel and loftness of Diapers 1-4 were tested by ranking the test diapers from worst (score: 1) to best (score: 4) in each test item of stiffness, grainy feel and loftness. The average score of scores from all the panelists is the final sensory score of the tested samples in Table 6.

TABLE 6

| | Diaper 1 | Diaper 2 | Diaper 3 | Diaper 4 |
|---|---|---|---|---|
| Stiffness | 4 | 2.25 | 1 | 2.75 |
| Grainy feel | 1.5 | 4 | 3.25 | 1.75 |
| Loftness | 1.5 | 4 | 3.25 | 2.25 |

Among Diapers 2-4 having 60 gsm intermediate layers, Diaper 2 were evaluated as significantly better in stiffness, grainy feel and loftness over Diaper 3, and significantly better grainy feel and loftness over Diaper 4.

The dimensions and values disclosed herein are not to be understood as being strictly limited to the exact numerical values recited. For example, a dimension disclosed as “40 mm” is intended to mean “about 40 mm ”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a topsheet, a backsheet, and a layer of absorbent material disposed between the topsheet and the backsheet, wherein the layer of absorbent material comprises superabsorbent polymer; and
an intermediate layer comprising a nonwoven web, the intermediate layer being disposed between the layer of absorbent material and the backsheet;
wherein the intermediate layer has a MD tensile/basis weight no greater than about 0.75 N/5 cm/g/m² as measured according to Tensile Strength Test, and a thickness/basis weight no less than about 0.078 mm/g/m², as measured FTT Test.

2. The absorbent article of claim 1, wherein the nonwoven web comprises first thermoplastic fibers having a first melting temperature and second thermoplastic fibers having a second melting temperature, and wherein a difference between the first melting temperature and the second melting temperature is at least about 40° C.

3. The absorbent article of claim 1, wherein the intermediate layer has a compression average rigidity no greater than about 400 gf/mm³ as measured according to FTT Test.

4. The absorbent article of claim 12, wherein the intermediate layer has a bending work no greater than about 2000 gf×mm×rad as measured according to FTT Test.

5. The absorbent article of claim 2, wherein the second thermoplastic fibers have a melting temperature greater at least about 40° C. than the first thermoplastic fibers.

6. The absorbent article of claim 2, wherein the second thermoplastic fibers are hollow fibers or shaped fibers.

7. The absorbent article of claim 5, wherein the first thermoplastic fibers are heat-fused one another.

8. The absorbent article of claim 5, wherein the second thermoplastic fibers are not heat-fused one another.

9. The absorbent article of claim 2, wherein the nonwoven web comprises third fibers.

10. The absorbent article of claim 1, wherein the intermediate layer comprises a nonwoven web selected from the group consisting of an air-through bonded nonwoven made of staple fibers, carded calendar bonded nonwovens made of staple fibers and combinations thereof, a spunlace nonwoven made of staple fibers, an air-through bonded nonwoven made of spunlaid fibers and a spunlace nonwoven made of spunlaid fibers.

11. The absorbent article of claim 2, wherein the nonwoven web comprises the first thermoplastic fibers of at least 40% by weight of the nonwoven web.

12. The absorbent article of claim 2, wherein the nonwoven web comprises the second thermoplastic fiber of at least 30% by weight of the nonwoven web.

13. The absorbent article of claim 1, further comprising an upper acquisition and distribution system with at least one layer, the upper acquisition and distribution system being disposed between the topsheet and the layer of absorbent material.

14. The absorbent article of claim 1, wherein the layer of absorbent material is partly or fully enclosed by and in direct contact with an upper substrate layer and a lower substrate layer, wherein the upper substrate layer is between the topsheet and the layer of absorbent material or between the upper acquisition and distribution system and the layer of absorbent material, and wherein the lower substrate layer is between the layer of absorbent material and the intermediate layer.

15. The absorbent article of claim 1, wherein the layer of absorbent material and the intermediate layer are partly or fully enclosed by and in direct contact with an upper substrate layer and a lower substrate layer, wherein the upper substrate is between the topsheet and the layer of absorbent material or between the upper acquisition and distribution system and the layer of absorbent material, and the lower substrate layer is between the intermediate layer and the backsheet, and wherein the lower substrate layer of absorbent material is in direct contact with the intermediate layer.

16. The absorbent article of claim 1, wherein the layer of absorbent material is partly or fully enclosed by and in direct contact with an upper substrate layer and the intermediate layer, and wherein the upper substrate layer is between the topsheet and the layer of absorbent material or between the upper acquisition and distribution system and the layer of absorbent material.

17. A nonwoven comprising a MD tensile/basis weight no greater than about 0.75 N/5 cm/g/m² as measured according to Tensile Strength Test, and a thickness/basis weight no less than about 0.078 mm/g/m², as measured FTT Test.

18. The nonwoven of claim 17, comprising first thermoplastic fibers having a first melting temperature and second thermoplastic fibers having a second melting temperature, wherein a difference between the first melting temperature and the second melting temperature is at least about 40° C., and wherein the second thermoplastic fibers are hollow fibers or shaped fibers.

19. A process for producing nonwoven comprising:
supplying a nonwoven web comprising first thermoplastic fibers having a first melting temperature and second thermoplastic fibers having a second melting temperature, wherein the second melting temperature is at least about 40° C. higher than the first melting temperature; and
applying heat to the nonwoven web so that at least part of the first thermoplastic fibers are heat-fused to one another, wherein the heat has a temperature between the first melting temperature and lower than the second melting temperature.

20. The process of claim 19, wherein the nonwoven has a MD tensile/basis weight no greater than about 0.75 N/5 cm/g/m² as measured according to Tensile Strength Test, and a thickness/basis weight no less than about 0.078 mm/g/m², as measured FTT Test.
